(19) United States
(12) Patent Application Publication
(10) Pub. No.: US 2006/0191536 A1
(43) Pub. Date: Aug. 31, 2006

(54) Bag mask resuscitator

(75) Inventors: Kevin D. Kroupa, Ballwin, MO (US); William Kullmann, Oakville, MO (US); Steve Palmer, Kirkwood, MO (US)

Correspondence Address: GREENSFELDER HEMKER & GALE PC, SUITE 2000, 10 SOUTH BROADWAY, ST LOUIS, MO, 63102, US

(73) Assignee: Allied Healthcare Products, Inc., St. Louis, MO (US)

(21) Appl. No.: 11/361,295

(22) Filed: Feb. 24, 2006

Related U.S. Application Data
(60) Provisional application No. 60/656,176, filed on Feb. 25, 2005.

(51) Int. Cl. A61M 16/00

(52) U.S. Cl. 128/205.13; 128/202.28; 128/205.17

(57) ABSTRACT

A bag mask resuscitator including flexible tubing having a proximal end and a distal end with the proximal end engaged to a resuscitation bag and the distal end adapted for engagement with a valve assembly is disclosed. The valve assembly includes a coupling member adapted for quick engagement and disengagement of the flexible tubing from the valve assembly using a simple action.

### CROSS-REFERENCE TO RELATED APPLICATION

This application claims the benefit of U.S. Provisional Patent Application Ser. No. 60/656,176, filed Feb. 25, 2005, which is herein incorporated by reference.

### FIELD

The present document relates to a.bag mask resuscitator, and more particularly to a bag mask resuscitator having a dual swivel arrangement and quick disconnect feature.

### SUMMARY

In one embodiment, the bag mask resuscitator may comprise a flexible tubing having a distal end and a proximal end, the distal end of the flexible-tubing defining at least one slot adapted to engage a valve assembly, while the proximal end of the flexible tubing being adapted to engage a resuscitation bag, and a mask being adapted to engage the valve assembly, the valve assembly including a coupling member having at least one pin adapted to engage or disengage the at least one slot of the flexible tubing.

In an embodiment, a bag mask resuscitator bag mask may include a flexible tubing having a distal end and a proximal end, the distal end of the flexible tubing defining at least one slot, the distal end of the flexible tubing being adapted to engage a valve assembly in a rotational relationship, while the proximal end of the flexible tubing being adapted to fixedly engage a flexible resuscitation bag, the valve assembly including a coupling member defining at least one pin adapted to engage or disengage the at least one slot of the flexible tubing, and a mask adapted to engage the valve assembly in a rotational relationship, the valve assembly further including a plurality of swivel retainers for providing a dual swivel arrangement that allows the valve assembly to freely rotate relative to the mask and/or the flexible tubing.

In another embodiment, a method of using a bag mask resuscitator may include:

a) providing a flexible tubing having a distal end and a proximal end, the distal end of the flexible tubing defining at least one slot adapted to engage a valve assembly, while the proximal end of the flexible tubing being adapted to fixedly engage a resuscitation bag, and a mask being adapted to engage the valve assembly, the valve assembly including a coupling member having at least one pin adapted to engage or disengage the at least one slot of the flexible tubing;

b) aligning the at least one pin of the valve assembly with the at least one slot of the flexible tubing;

c) engaging the at least one pin of the valve assembly with the at least one slot of the flexible tubing; and

d) rotating the at least one pin in a direction relative to the at least one slot such that the flexible tubing becomes engaged to the valve assembly.

Implementation of the above embodiments may include one or more of the following features:

The valve assembly may include a swivel retainer adapted to engage the mask in a rotational relationship.

The valve assembly may include a swivel retainer adapted to engage the flexible tubing in a rotational relationship.

The valve assembly may include a dual swivel arrangement having an swivel retainer adapted to engage the mask in a rotational relationship and another swivel retainer adapted to engage the flexible tubing in a rotational relationship such that the valve assembly freely rotates relative to the mask and/or the flexible tubing.

The valve assembly may include a dual swivel arrangement that permits the valve assembly to swivel along different planes relative to the mask and the flexible tubing.

The valve assembly further may include a one-way valve for regulating fluid flow communication.

The flexible tubing may be of sufficient length and flexibility that any forces generated by the resuscitation bag are not translated to the mask through the valve assembly.

The flexible tubing may be hollow such that actuation of the resuscitation bag forces air into the flexible tubing and through the valve assembly.

The valve assembly may include a top casing engaged to a bottom casing defining a chamber in communication with an inlet port adapted to engage the flexible tubing in a rotational relationship and an outlet port adapted to engage the mask in a rotational relationship.

The top casing and the bottom casing encases a one way duck bill valve that prevents back flow of air back through the flexible tubing a The bottom casing includes a ridged connector with the ridged connector adapted to engage a deflector.

At least one slot is a J-shaped slot.

At least one slot defines an entrance in communication with a terminal end portion.

The mask may include a port with the port adapted to be in a rotational relationship with one of the plurality of swivel retainers.

The flexible tubing may be adapted to engage the valve assembly in a rotational relationship with one of the plurality of swivel retainers.

The method further includes rotating the at least one pin in an opposite direction relative to the at least one slot, and disengaging the at least one pin from the at least one slot such that the flexible tubing becomes disengaged from the valve assembly.

Aligning the at least one pin with the at least one slot requires aligning the at least one pin with the entrance of the at least one slot.

Additional features will be set forth in the description which follows or will become apparent to those skilled in the art upon examination of the drawings and detailed description which follows.

### BRIEF DESCRIPTION OF THE DRAWINGS

FIG. 1 is a side view of the bag mask resuscitator;

FIG. 2 is a top view of the bag mask resuscitator;

FIG. 3 is an exploded view of a resuscitation bag for the bag mask resuscitator;

FIG. 4 is an end view of the resuscitation bag;

FIG. 5 is a cross-sectional view of the resuscitation bag taken along line 5-5 of FIG. 4;

FIG. 6 is an orthogonal view of an outlet manifold for the resuscitation bag;

FIG. 7 is an end view of the outlet manifold;

FIG. 8 is a cross-sectional view of the outlet manifold taken along line 8-8 of FIG. 7;

FIG. 9 is an enlarged cross-sectional view of the outlet manifold taken along line 9-9 of FIG. 7;

FIG. 10 is an orthogonal view of a check valve for the resuscitation bag;

FIG. 11 is a side view of the check valve;

FIG. 12 is an orthogonal view of a pressure relief valve for the resuscitation bag;

FIG. 13 is a side view of the pressure relief valve;

FIG. 14 is another side view of the pressure relief valve;

FIG. 15 is an exploded view of a valve assembly for the bag mask resuscitator;

FIG. 16 is an orthogonal view of the valve assembly;

FIG. 17 is an end view of the valve assembly;

FIG. 18 is a cross-sectional view of the valve assembly taken along line 18-18 of FIG. 17;

FIG. 19 is an orthogonal view of a mask for the bag mask resuscitator;

FIG. 20 is a cross-sectional view of one embodiment of the resuscitation bag; and

FIG. 21 is a partial cross-sectional view of the coupling member illustrating the engagement with the distal end of the flexible, tubing.

Corresponding reference characters indicate corresponding elements among the view of the drawings.

### DETAILED DESCRIPTION

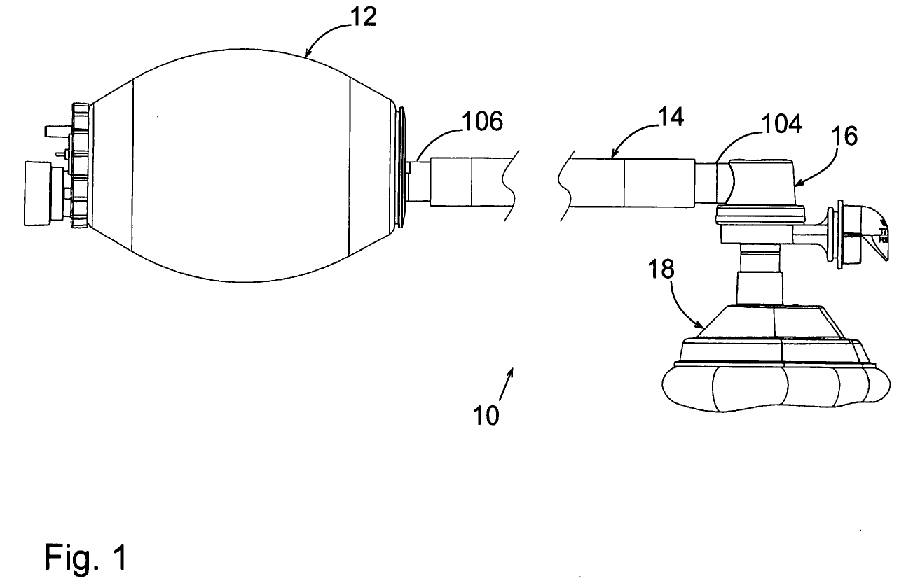

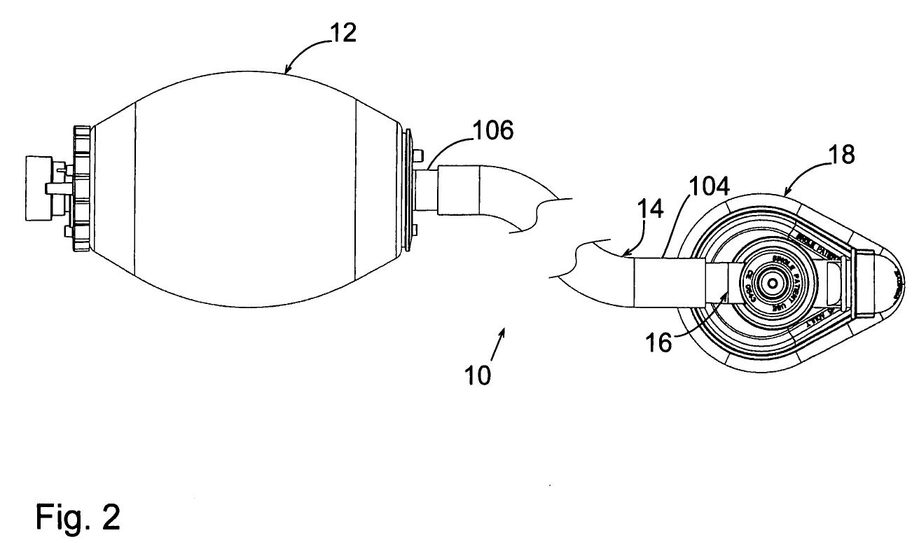

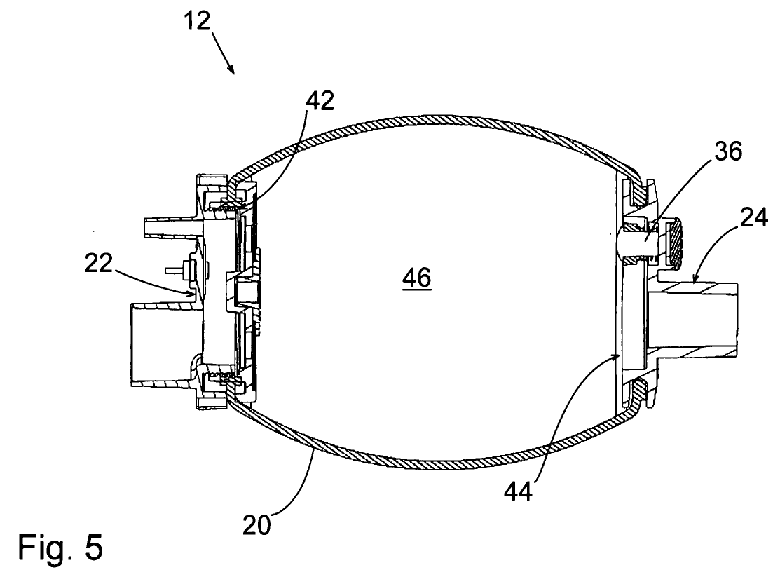

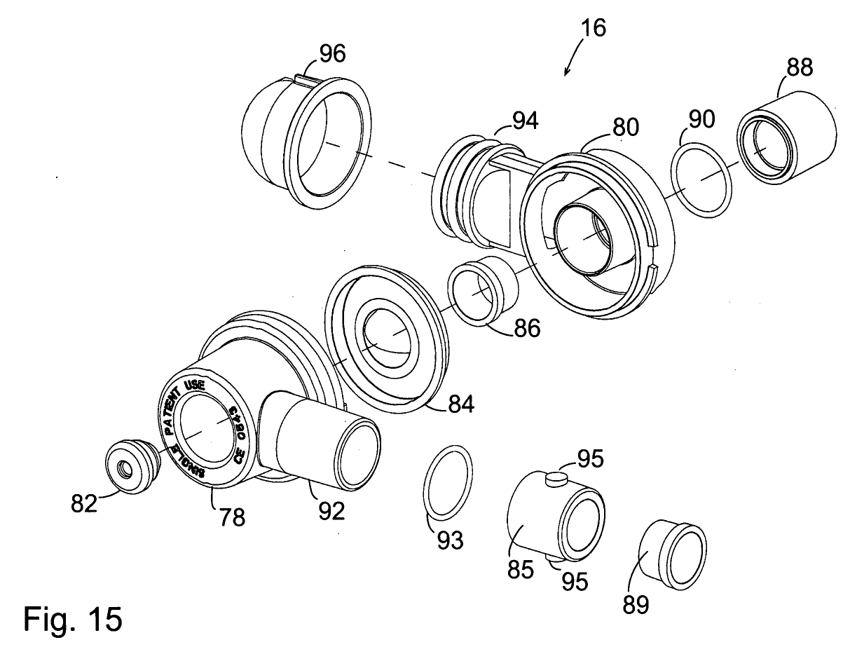

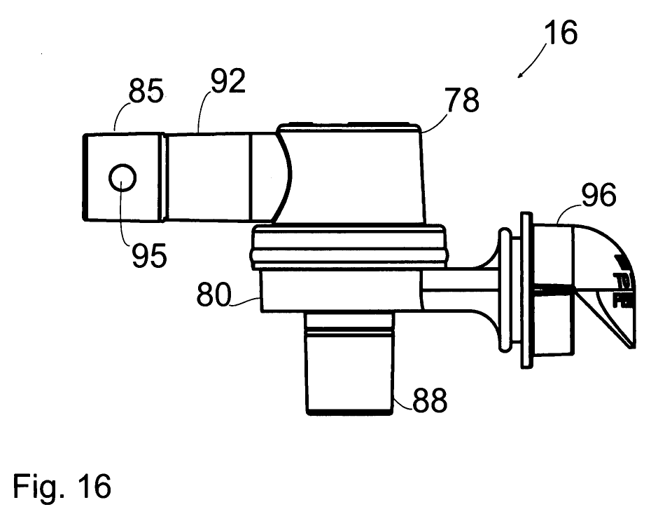

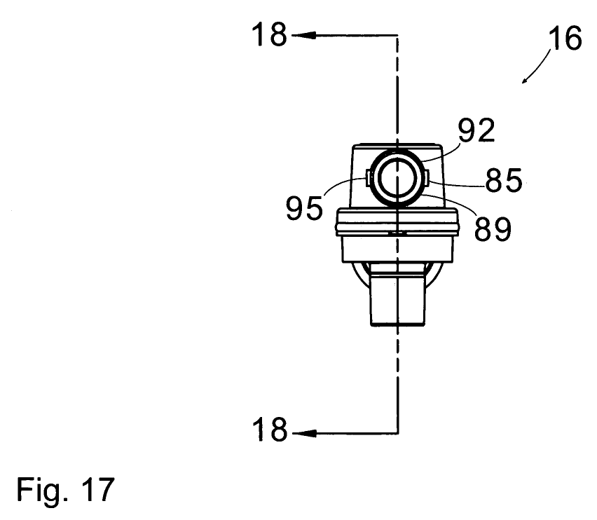

Referring to the drawings, an embodiment of the bag mask resuscitator is illustrated and generally indicated as 10 in FIGS. 1-21. As shown in FIGS. 1 and 2, bag mask resuscitator 10 may include a hollow, flexible tubing 14 of suitable length and flexibility having a proximal end 106 fixedly engaged to a resuscitation bag 12 and a distal end 104 adapted to be engaged to a valve assembly 16 in a rotational relationship. In addition, valve assembly 16 may be adapted to engage a mask 18 in a rotational relationship such that a dual swivel arrangement is provided by the valve assembly 16. As used herein, the term “distal” means the end farthest from the user, while “proximal” means the end closest to the user.

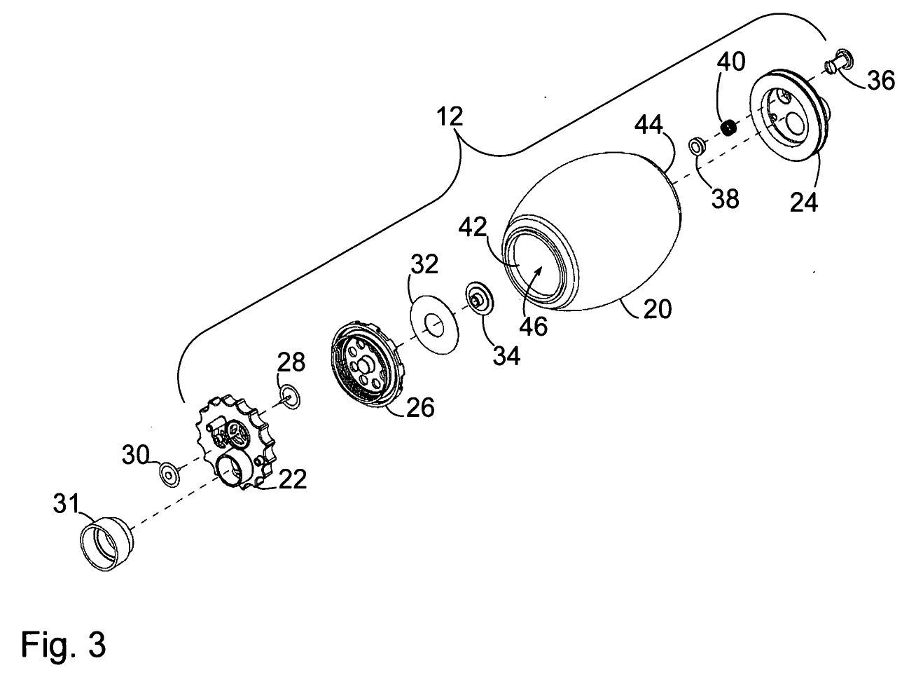

Referring to FIGS. 3-5, resuscitation bag 12 provides a means for forcing air into a patient's respiratory system by repeated actuation and includes a flexible, hollow body 20 defining a chamber 46 that communicates with a proximal opening 42 and a distal opening 44. An inlet manifold 22 and an outlet manifold 24 may be disposed along the proximal and distal openings 42, 44, respectively, for acting as a connection site as well as regulating fluid flow through the resuscitation bag 12. The resuscitation bag 12 may further include a main valve flapper 32 engaged to a main valve retainer 34 that regulates the direction of fluid flow through a retainer 26 when the resuscitation bag 12 is actuated.

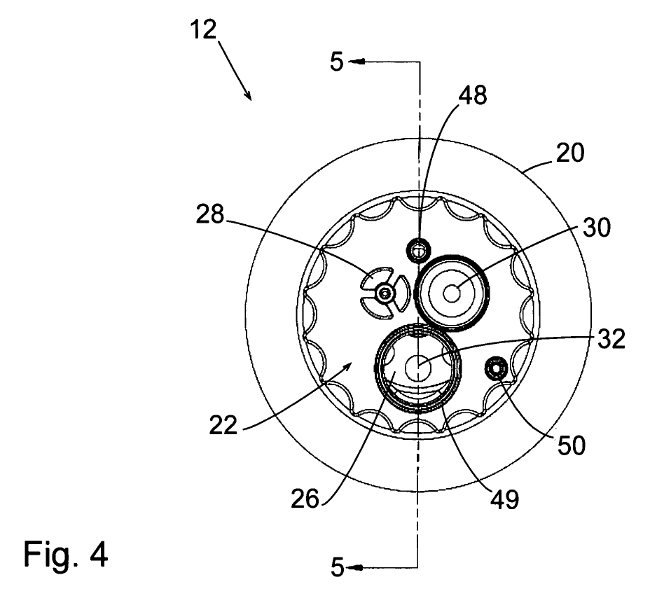

As shown specifically in FIGS. 3 and 4, the inlet manifold 22 may be engaged to proximal opening 42 by retainer 26 and may define a plurality of ports. The inlet manifold 22 may further include a first check valve 28 and a second check valve 30 for regulating the fluid flow communication with atmosphere. Both first and second check valves 28 and 30 may be similar conventional one-way check valves with the first check valve 28 only permitting fluid flow into the chamber 46 created by the joining of inlet manifold 22 and retainer 26, while second check valve 30 only permits fluid flow out from the chamber 46 created by the joining of inlet manifold 22 and retainer 26, thereby regulating fluid flow through the inlet manifold 22.

When resuscitation bag 12 is actuated by compressing body 20, main valve flapper 32 is closed and permits air to be forced through tubing 14. Conversely, when the resuscitation bag 12 is decompressed the main valve flapper 32 and the first check valve 28 are open to fluid flow and the second check valve 30 is closed to permit incoming air from atmosphere to re-fill chamber 46.

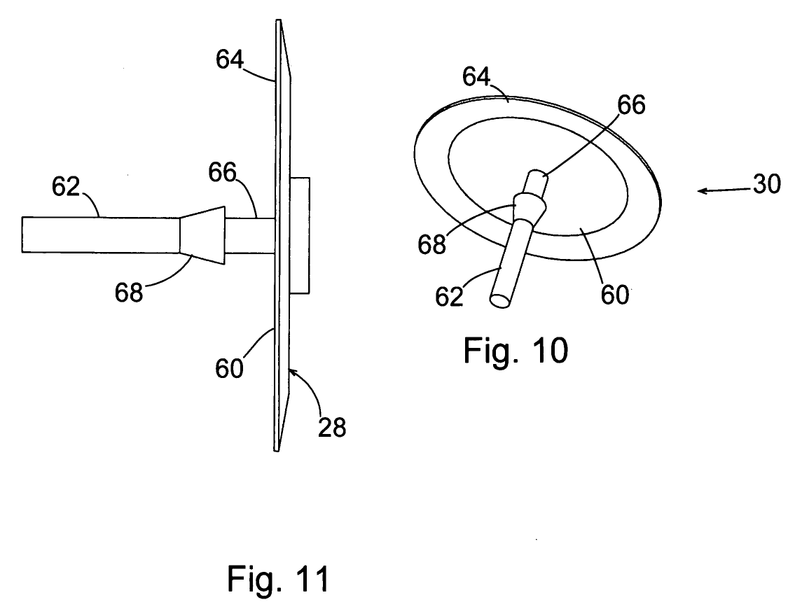

Referring to FIGS. 10 and 11, both first and second check valves 28, 30 may have the same structure and include an umbrella 60 defining a peripheral portion 64 and a stem 62 extending axially from the center of umbrella 60. Stem 62 may define a shoulder 68 with a neck 66 interposed between the shoulder 68 and umbrella 60. Both first and second check valves 28, 30 permit one way fluid flow communication through the resuscitation bag 12.

Referring to FIG. 4, inlet manifold 22 may further include a reservoir bag inlet 49 for attachment to a reservoir bag (not shown), an oxygen inlet 48 adapted to be operatively engaged to a source of oxygen for providing supplemental oxygen to the patient through resuscitation bag 12, and a boss 50 defined for receiving a retaining pin (not shown) adapted to engage a handle (not shown) for handling resuscitation bag 12 by a user. In addition, an adaptor 31 (FIG. 3) may be engaged to the reservoir bag inlet 49 for engagement to the reservoir bag (not shown). The reservoir bag may act as a temporary storage site for collecting excess oxygen supplied by the oxygen source during operation of bag mask resuscitator 10.

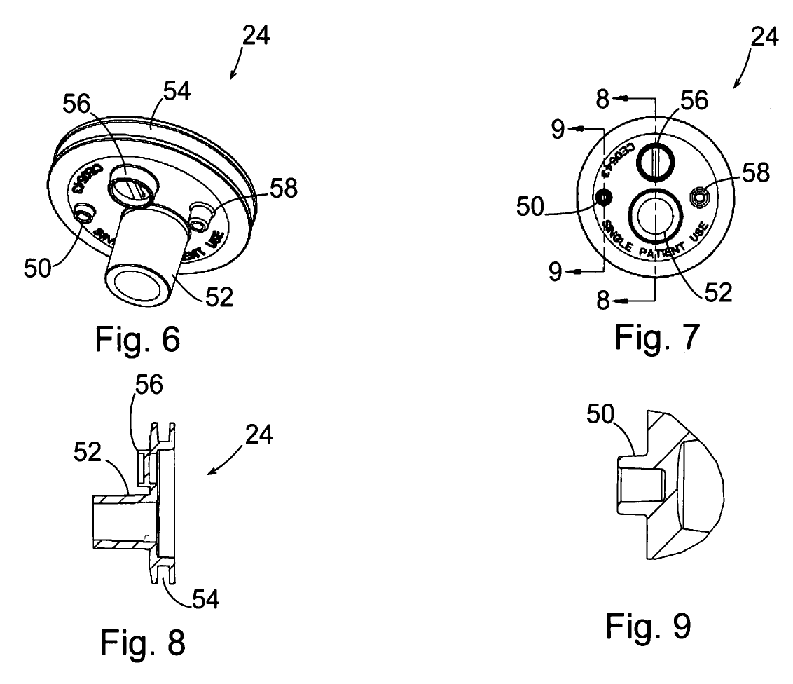

Referring to FIGS. 6-9, the outlet manifold 24 may define a peripheral channel 54 adapted to engage the distal opening 44 of resuscitation bag 12 as well as a plurality of ports. The outlet manifold 24 may include an outlet port 52 adapted to engage the flexible tubing 14 to the resuscitator bag 12 and a pressure relief valve port 56 adapted to receive a pressure relief valve 36 (FIG. 3) for venting excess pressure if the internal pressure exceeds a predetermined threshold. In addition, a manometer port 58 may be adapted to receive a manometer (not shown) for indicating internal pressure as well as a boss 50 that engages a retaining pin (not shown) used to secure one end of the handle that may be engaged to resuscitation bag 12 at the outlet manifold 24.

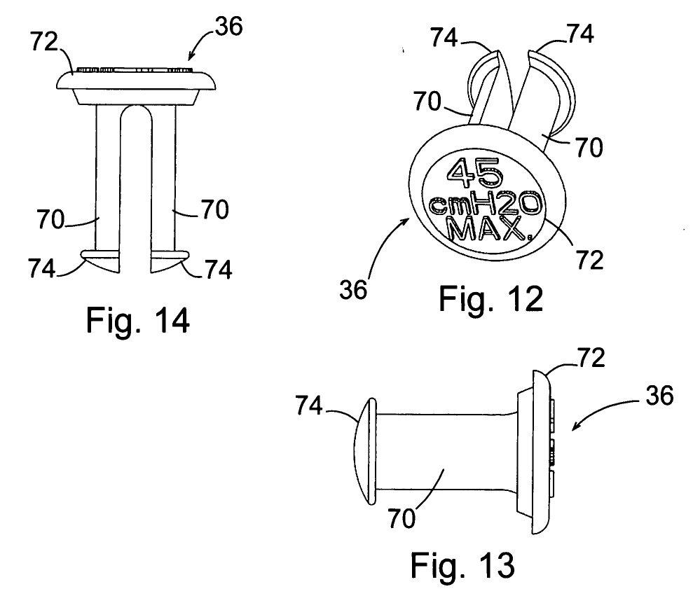

Referring to FIGS. 12, 13 and 14, pressure relief valve 36 may include a top portion 72 defining a pair of legs 70 with a tang 74 formed at the free end of each leg 70 adapted to operatively engage a pressure relief valve retainer 38 (FIG. 3). A spring 40 (FIG. 3) is provided that applies a spring force that maintains the pressure relief valve 36 in a normally-closed position and prevents fluid flow communication. When an excess pressure condition occurs that overcomes the spring force of the spring 40 the pressure relief valve 36 is placed in an open position that permits the excess pressure to vent to atmosphere. The pressure relief valve 36 then returns to the normally-closed position after the pressure falls below the predetermined threshold. In one embodiment, the pressure relief valve 36 may have a predetermined threshold set at 45 cmH2O, although other predetermined thresholds are contemplated.

The valve assembly 16 may regulate fluid flow communication between the flexible tubing 14 and the mask 18 while also providing a dual swivel arrangement for isolating forces generated by actuation of the resuscitation bag 12 that might affect the placement of the mask 18 on the patient's face.

Referring to FIGS. 15-18, valve assembly 16 may include a top casing 78 engaged to a bottom casing 80 that collectively define a chamber 79 encasing a one-way duck bill valve 84 that prevents the back flow of air back through flexible tubing 14.

In particular, the top casing 78 may define an inlet port 92 having a coupling member 85 in swivel connection with the flexible tubing 14 through a swivel retainer 89. The swivel retainer 89 forms a part of the dual swivel arrangement which permits the flexible tubing 14 to swivel or rotate relative to valve assembly 16 along a specific plane.

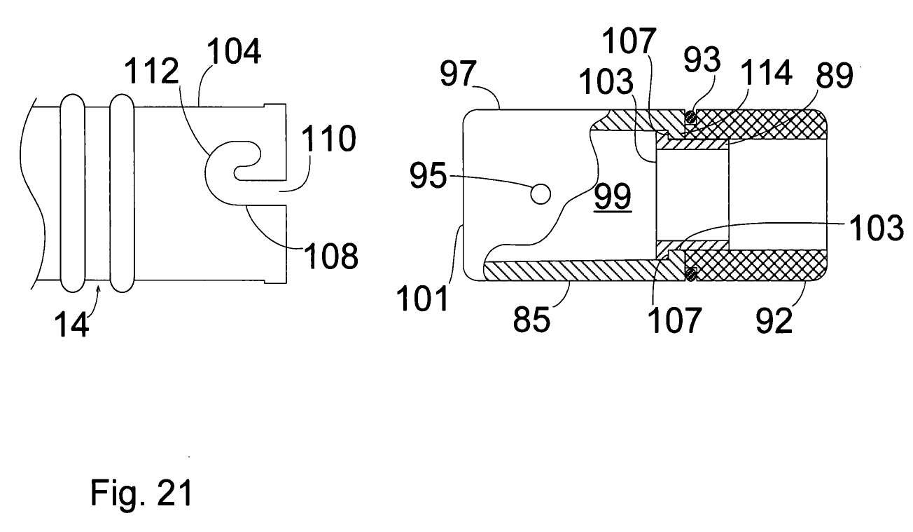

Referring to FIG. 21, the coupling member 85 includes a body 97 that defines a conduit 99 in communication with a distal opening 101 and a proximal opening 103. The proximal opening 101 may define an outer shoulder 107 adapted to abut a flange 114 defined by swivel retainer 89. A sealing element 93 may be provided between the coupling member 85 to provide an air-tight seal. Coupling member 85 further includes a pair of opposing pins 95 that forms a part of a quick connect or disconnect feature adapted to engage or disengage the flexible tubing 14 from the valve assembly 16 as shall be discussed below.

As further shown, the distal end 104 of flexible tubing 14 may define a pair of J-shaped slots 108 adapted to engage respective opposing pins 95 of coupling member 85 and forms a part of the quick connect feature when engaging the flexible tubing 14 to the valve assembly 16. Each J-shaped slot 95 defines an entrance 110 in communication with a terminal end portion 112. To provide the quick connect feature between the flexible tubing 14 and the valve assembly 16, the user aligns the opposing pins 95 with a respective entrance 110 of J-shaped slot 108 and the user then pushes the flexible tubing 14 toward the valve assembly 16 such that each pin 95 enters through a respective entrance 110. The user then rotates the flexible tubing 14 in one direction relative to valve assembly 16 such that each pin 95 becomes fully engaged with the terminal end portion 112 of each J-shaped slot 108.

Conversely, to provide a quick disconnect feature between the flexible tubing 14 and valve assembly 16, the user rotates the flexible tubing 14 in an opposite direction such that pins 95 disengage from each respective terminal end portion 112. The user then pulls axially on the flexible tubing 14 away from valve assembly 16 until the pins 95 disengage from each respective entrance 110 and the flexible tubing 14 disengages from valve assembly 16.

In addition, top casing 78 of valve assembly 16 may include an injection port 82, such as a rubber bung, adapted to receive a syringe or like device for injecting fluid, such as medicine, into the patient's respiratory system through mask 18.

The bottom casing 80 may include an outlet port 88 engaged to a swivel retainer 86 that is adapted to engage the mask 18 in a rotational relationship similar to the swivel connection of the inlet port 92 with flexible tubing 14 which permits the valve assembly 16 to freely rotate relative to mask 18. As such, the swivel retainer 89 of inlet port 92 and the swivel retainer 86 of outlet port 88 provide a dual swivel arrangement that permits the valve assembly 16 to swivel along two different planes, thereby permitting the valve assembly 16 to freely rotate relative to mask 18 and/or flexible tubing 14.

In one embodiment, flexible tubing 14 may be of sufficient length and flexibility that, in combination with the dual swivel arrangement of the valve assembly 16, forces generated by actuation of the resuscitation bag 12 are isolated and are not applied to the mask 18. This is because the flexible tubing 14 and the dual swivel arrangement of the valve assembly 16 effectively prevent such forces from affecting the placement of mask 18 on the patient's face. In other words, the length and flexibility of flexible tubing 14 assists in preventing forces generated by the resuscitation bag 12 to be translated to the valve assembly 16, and any forces which may be translated to the valve assembly 16 are effectively blocked from being translated to mask 18 by the dual swivel arrangement of the valve assembly 16.

As further shown, bottom casing 80 of the valve assembly 16 may include a ridged connector 94 adapted to engage a deflector 96 that permits any breath that might contain contaminants expelled by the patient to be safely deflected away from the healthcare practitioner. If so desired, the deflector 96 may be removed and replaced with a PEEP valve (not shown) that permits retention of a small amount of air pressure inside the respiratory system of the patient in order to prevent the lungs from collapsing. A sealing element 90 may be provided between the outlet port 88 and bottom casing 80 for providing an air-tight seal.

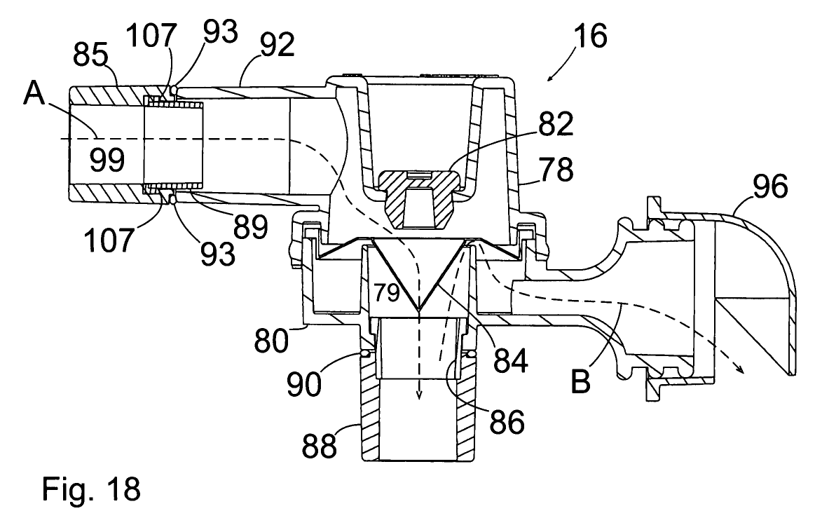

As illustrated in FIG. 18, actuation of the resuscitation bag 12 forces air along a path, represented by fluid pathway A, through flexible tubing 14 and into the inlet port 92 of the valve assembly 16. The forced air then passes through the duck bill valve 84 and exits out from outlet port 88 where the forced air enters the patient's respiratory system. When the patient exhales, the expelled breath enters back through the outlet port 88 and follows a fluid pathway B which exits the valve assembly 16 at deflector 96.

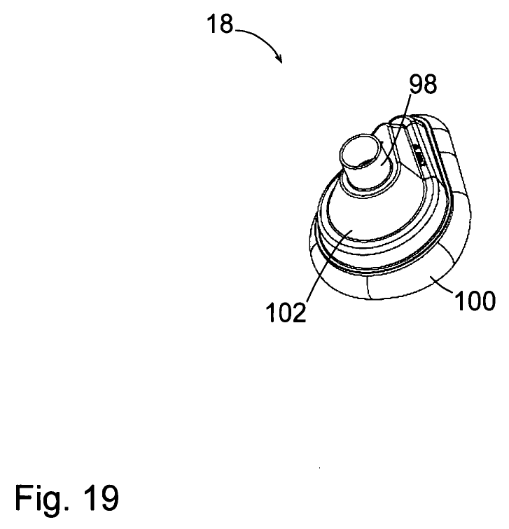

Referring to FIG. 19, mask 18 may include a mask portion 102 defining a port 98 adapted to engage outlet port 88 of valve assembly 16 such that the valve assembly 16 freely rotates relative to port 98 of mask 18 along a specific plane, thereby isolating any forces applied to valve assembly 16 by flexible tubing 14 such that any exterior forces are prevented from being applied to mask 18. The mask 18 may further include seal portion 100 defined along the periphery of mask portion 102 to provide an air tight seal between the mask 18 and the patient's face.

In one embodiment, a method for using the bag mask resuscitator 10 is contemplated. The method may include securing the mask 18 to the patient's face, positioning the resuscitation bag 12 remotely away from the patient, and actuating the resuscitation bag 12 between the user's hip and single hand such that the forces generated by actuation of the resuscitation bag 12 are not applied or translated to the mask 18 due to the length of flexible tubing 14 in combination with the dual swivel arrangement of the valve assembly 16.

In this particular embodiment, the resuscitation bag 12 may be positioned remotely away from the patient in a manner that permits actuation of the resuscitation bag 12 without interfering with other healthcare personnel who may be directly attending to the patient. As such, flexible tubing 14 may be of sufficient length to permit the resuscitation bag 12 to be operated in a range of 1-2 feet from the patient's face without disturbing the placement of mask 18 during actuation of resuscitation bag 12 or interfere with other attending healthcare personnel. However, longer lengths of flexible tubing 14 are contemplated.

In another embodiment, the method may include securing the mask to the patient's face, positioning the resuscitation bag 12 remotely from the patient and between a user's two hands, and actuating the resuscitation bag 12 between the two hands of the user such that the forces generated by actuation of the resuscitation bag 12 are not applied or translated to the mask 18 as noted above.

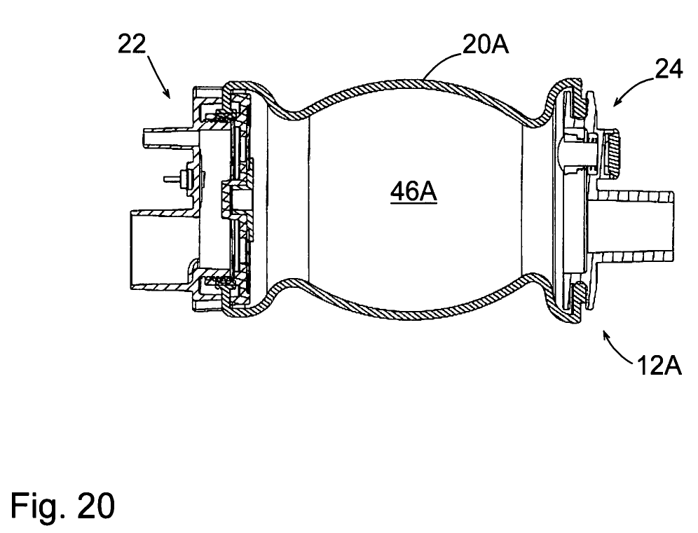

In yet another embodiment, the resuscitation bag, designated 12A, may be adapted for pediatric applications as shown in FIG. 20. The resuscitation bag 12A may include the same inlet manifold 22 and outlet manifold 24 and functions in the same manner as the other embodiment except body 20A has a different configuration and defines a chamber 46A having a smaller volume than chamber 46 of the resuscitation bag 12.

The bag mask resuscitator 10 may be used with an endotracheal tube (not shown) by connecting the outlet port 88 of the valve assembly 16 to the end of the endotracheal tube. In this manner a resuscitation procedure that forces air into the patient's respiratory system through the valve assembly 16 from the resuscitation bag 12 may be accomplished.

It should be understood from the foregoing that, while particular embodiments have been illustrated and described, various modifications can be made thereto without departing from the spirit and scope of the invention as will be apparent to those skilled in the art. Such changes and modifications are within the scope and teaching of this invention as defined in the claims appended hereto.

1. A bag mask resuscitator comprising:
a flexible tubing having a distal end and a proximal end, said distal end of said flexible tubing defining at least one slot adapted to engage a valve assembly, while said proximal end of said flexible tubing being adapted to engage a resuscitation bag, and
a mask being adapted to engage said valve assembly, said valve assembly including a coupling member having at least one pin adapted to engage or disengage said at least one slot of said flexible tubing.

2. The bag mask resuscitator according to claim 1, wherein said valve assembly comprises a swivel retainer adapted to engage said mask in a rotational relationship.

3. The bag mask resuscitator according to claim 1, wherein said valve assembly comprises a swivel retainer adapted to engage said flexible tubing in a rotational relationship.

4. The bag mask resuscitator according to claim 1, wherein said valve assembly includes a dual swivel arrangement having an swivel retainer adapted to engage said mask in a rotational relationship and another swivel retainer adapted to engage said flexible tubing in a rotational relationship such that said valve assembly freely rotates relative to said mask and/or said flexible tubing.

5. The bag mask resuscitator according to claim 2, wherein said valve assembly includes a dual swivel arrangement that permits said valve assembly to swivel along different planes relative to said mask and said flexible tubing.

6. The bag mask resuscitator according to claim 1, wherein said valve assembly further includes a one-way valve for regulating fluid flow communication.

7. The bag mask resuscitator according to claim 1, wherein said flexible tubing is of sufficient length and flexibility that any forces generated by said resuscitation bag are not translated to said mask through said valve assembly.

8. The bag mask resuscitator according to claim 1, wherein said flexible tubing is hollow such that actuation of said resuscitation bag forces air into said flexible tubing and through said valve assembly.

9. The bag mask resuscitator according to claim 1, wherein said valve assembly includes a top casing engaged to a bottom casing defining a chamber in communication with an inlet port adapted to engage said flexible tubing in a rotational relationship and an outlet port adapted to engage said mask in a rotational relationship.

10. The bag mask resuscitator according to claim 9, wherein said top casing and said bottom casing encases a one way duck bill valve that prevents back flow of air back through said flexible tubing.

11. The bag mask resuscitator according, to claim 9, wherein said bottom-casing includes a ridged connector, said ridged connector adapted to engage a deflector.

12. The bag mask resuscitator according to claim 1, wherein said at least one slot is a J-shaped slot.

13. The bag mask resuscitator according to claim 12, wherein each of said at least one slot define an entrance in communication with a terminal end portion.

14. A bag mask resuscitator comprising:
a flexible tubing having a distal end and a proximal end, said distal end of said flexible tubing defining at least one slot, said distal end of said flexible tubing being adapted to engage a valve assembly in a rotational relationship, while said proximal end of said flexible tubing being adapted to fixedly engage a flexible resuscitation bag, said valve assembly including a coupling member defining at least one pin adapted to engage or disengage said at least one slot of said flexible tubing, and
a mask adapted to engage said valve assembly in a rotational relationship, said valve assembly further including a plurality of swivel retainers for providing a dual swivel arrangement that allows said valve assembly to freely rotate relative to said mask and/or said flexible tubing.

15. The bag mask resuscitator according to claim 14, wherein said mask includes a port, said port adapted to be in a rotational relationship with one of said plurality of swivel retainers.

16. The bag mask resuscitator according to claim 15, wherein said flexible tubing being adapted to engage said valve assembly in a rotational relationship with one of said plurality of swivel retainers.

17. The bag mask resuscitator according to claim 14, wherein said at least one slot defines an entrance in communication with a terminal end portion.

18. A method of using a bag mask resuscitator comprising:
a) providing a flexible tubing having a distal end and a proximal end, said distal end of said flexible tubing defining at least one slot adapted to engage a valve assembly, while said proximal end of said flexible tubing being adapted to fixedly engage a resuscitation bag, and a mask being adapted to engage said valve assembly, said valve assembly including a coupling member having at least one pin adapted to engage or disengage said at least one slot of said flexible tubing;
b) aligning said at least one pin of said valve assembly with said at least one slot of said flexible tubing;
c) engaging said at least one pin of said valve assembly with said at least one slot of said flexible tubing; and
d) rotating said at least one pin in a direction relative to said at least one slot such that said flexible tubing becomes engaged to said valve assembly.

19. The method according to claim 18, further comprising:
e) rotating said at least one pin in an opposite direction relative to said at least one slot; and
f) disengaging said at least one pin from said at least one slot such that the flexible tubing becomes disengaged from said valve assembly.

20. The method according to claim 18, wherein each of said at least one slot defines an entrance in communication with a terminal end portion.

21. The method according to claim 20, wherein aligning said at least one pin with said at least one slot requires aligning said at least one pin with said entrance of said at least one slot.
